(19) United States
(12) Patent Application Publication
(10) Pub. No.: US 2024/0149010 A1
(43) Pub. Date: May 9, 2024

(54) SLEEP-INDUCING ELECTRONIC DEVICE

(71) Applicant: Gregory LEKHTMAN, Dorval (CA)

(72) Inventor: Gregory LEKHTMAN, Dorval (CA)

(21) Appl. No.: 18/387,267

(22) Filed: Nov. 6, 2023

Related U.S. Application Data
(60) Provisional application No. 63/423,698, filed on Nov. 8, 2022.

(51) Int. Cl. A61M 21/02

(52) U.S. Cl. CPC A61M 21/02; A61M 2021/0044

(57) ABSTRACT

A sleep-inducing electronic device, for being in skin contact with a user to operate, includes an input unit with first and second sensors detecting physiological signals of the user. A processor unit operatively connects to the sensors to receive the detected physiological signals therefrom and monitors at least one physiological parameter from a parameter group consisting of an electromyogram (EMG) signal and a galvanic skin response (GSR) signal. The processor unit converts the at least one physiological parameter into light stimulus signal parameters. An output unit connected to the processor unit to receive the light stimulus signal parameters therefrom produces a corresponding light stimulus signal for the user.

### CROSS-REFERENCE TO RELATED APPLICATIONS

This application claims priority of U.S. Provisional Application for Patent No. 63/423,698 filed Nov. 8, 2022, the content of which is incorporated herein in its entirety by reference.

### FIELD OF THE INVENTION

The present invention relates to electronic devices, and more particularly to an electronic device that induces sleep to the user via a modulated light stimulus signal.

### BACKGROUND OF THE INVENTION

In order to have a good sleep, the brain activity of a person should be able to tend toward a ‘relax’ mode. In such a case, sleep is triggered by the nervous system controlled by electrophysiological brain activity (waves) in relationship to the transition of brain waves from alpha (α) to beta (ß) to theta (θ) waves.

Nowadays, however, with the increasing stress conditions affecting people in their day-to-day life, some people have great difficulty to get a good rest or even fall asleep. There exist pills or the like medicine to help people sleep, but these are invasive solutions that might have adverse effect(s) on a long run.

It is well known that electronic devices such as computers, smartphones and the like that excites the brain instead of allowing it to enter the sleep mode when needed. Consequently, some manufacturers automatically (or allow to) change the screen color of these devices, with less ‘cold’ colors, at specific times of the day (evening and night) to help removing the negative effects on the sleep.

There also exist electronic devices that produces either an electric contact signal through the skin or an audio signal for the ears that enhances the relaxation and improves sleep of the user. But in these devices, a sleep-inducing light stimuli is not controlled by the electrophysiology of the brain and body with non-invasive simple-to-use monitoring of the sleep electrophysiology by a portable self-contained, battery-operated device.

Accordingly, there is a need for an improved sleep-inducing electronic device.

### SUMMARY OF THE INVENTION

It is therefore a general object of the present invention to provide an improved sleep-inducing electronic device that obviates the above-mentioned drawbacks.

An advantage of the present invention is that the device is convenient, easy-to-use, preferably self-content, with no adjustment required and psychologically and physiologically inert. The device is preferably an all-inclusive one-part body housing, although it could be separated into multiple discrete components. Also, the device main component is preferably shaped to comfortably fit the palm of a hand of the user, although it could also be adapted to other body peripherals of the user. Preferably, the housing is cylindrical in shape, although any other shape could be suitable and convenient depending on the application.

Another advantage of the present invention is that the device typically has an automatic on/off activation for convenience of use as well as battery preservation. When the user holds the device, the detection of physiological activity turns on the device. The device automatically turns off when the user releases the device, either voluntarily or simply when falling asleep.

A further advantage of the present invention is that the device has a waterproof and/or shock resistant housing to prevent breakage.

Still another advantage of the present invention is that the device is not operatively affected by external (environmental) electromagnetic radiation (EER) and body movement of the user.

Yet another advantage of the present invention is that the device is sensitive to neurophysiological parameters in the user's brain to adequately control the light stimulus signal that will induce sleep of the user. The light stimulus signal produces alpha (α) and theta (θ) waves activity in the electroencephalogram (EEG) of the user's brain which automatically correlates to electromyogram (EMG) and/or galvanic skin response (GSR) and/or heart rate (HR) activity in the palm (or other body extremities) of the user's hand monitored in real-time by the device.

Yet a further advantage of the present invention is that the device provides for real-time surface monitoring of physiological signals such as electromyogram (EMG) signal and/or galvanic skin response (GSR) signal, with or without combination with a heart rate (HR) signal, to produce a modulated light stimulus signal that induces sleep to the user. The modulated light stimulus signal is correlated (customized and/or calibrated) with sleeping brain waves of the user that are associated with brain sleeping pattern. The modulated light stimulus signal typically induces three successive (over time) stages of sleep by color light stimulation controlled by electrophysiological signals from the user that are related to the above-described brain activity when triggering sleep.

Still another advantage of the present invention is that the device senses physiological signals via at least two, but preferably three, electrically conductive surfaces of sensors or detectors. The sensors are connected to a processor that generates a modulated visual light stimulus signal. A calibration of the required light stimulus signal based on the electrocardiogram (ECG) correlation to light stimuli parameters (frequency of the color(s), frequency of the stimulus signal, and amplitude/brightness of the stimulus signal) is typically performed in factory. When colors and frequencies are selected or chosen, there is no need for calibration.

Yet another advantage of the present invention is that the device is typically totally (entirely) automatic and does not require any knowledge to operate it.

Still a further advantage of the present invention is that the device can be used effectively by adults and/or children.

A further advantage of the present invention is that the device is safe and non-invasive.

Yet a further advantage of the present invention is that the device operates in harmony with natural sleep pattern.

According to an aspect of the present invention, there is provided a sleep-inducing electronic device for being in skin contact with a user, the device comprising:
an input unit including first and second sensors detecting physiological signals of the user;
a processor unit operatively connecting to the first and second sensors to receive the detected physiological signals therefrom, the processor unit monitoring at least one physiological parameter from a parameter group consisting of an electromyogram (EMG) signal and a galvanic skin response (GSR) signal, the processor unit converting the at least one physiological parameter into light stimulus signal parameters; and
an output unit operatively connecting to the processor unit to receive the light stimulus signal parameters therefrom and producing a corresponding light stimulus signal for the user.

In one embodiment, the parameter group further including a heart rate (HR) signal, and the at least one physiological parameter further includes the heart rate (HR) signal.

In one embodiment, the input unit further includes a third sensor operatively connecting to the processor unit and either detecting physiological signals of the user or being a neutral ground electrode.

Conveniently, the light stimulus signal parameters include a frequency parameter, an amplitude parameter and a color parameter.

In one embodiment, the processor unit timely modulating the light stimulus signal parameters to improve sleeping brain waves of the user associated with a brain sleeping pattern.

Conveniently, the processor unit timely modulates the light stimulus signal parameters over three successive stages of sleep of the user.

In one embodiment, the processor unit balancing detected physiological signals to reject artefact noise from at least one of external electromagnetic radiations (EMR) and body movements of the user.

In one embodiment, the processor unit controlling operation of the device upon detection of the user's physiological signals.

In one embodiment, the light stimulus signal induces production of alpha (α), beta (ß) and theta (θ) waves activity in the user's brain electroencephalogram (EEG) detectable by the controller unit in the at least one physiological parameter.

In one embodiment, the input unit, the processor unit, and the output unit are within a same housing.

In one embodiment, the third sensor is a neutral ground electrode.

In one embodiment, the third sensor is an infrared sensor, and

preferably an infrared opto-electronic sensor.

Conveniently, the device includes a power source located within the housing and electrically connected to the controller unit for operation of the device.

In one embodiment, the device includes a power source electrically connected to the controller unit for operation of the device.

Other objects and advantages of the present invention will become apparent from a careful reading of the detailed description provided herein, with appropriate reference to the accompanying drawings.

### BRIEF DESCRIPTION OF THE DRAWINGS

Further aspects and advantages of the present invention will become better understood with reference to the description in association with the following Figures, in which similar references used in different Figures denote similar components, wherein:

FIG. 1 is a schematic perspective view of a sleep-inducing electronic device in accordance with an embodiment of the present invention; and

FIG. 2 is a schematic block diagram of a sleep-inducing electronic device in accordance with an embodiment of the present invention.

### DETAILED DESCRIPTION OF THE INVENTION

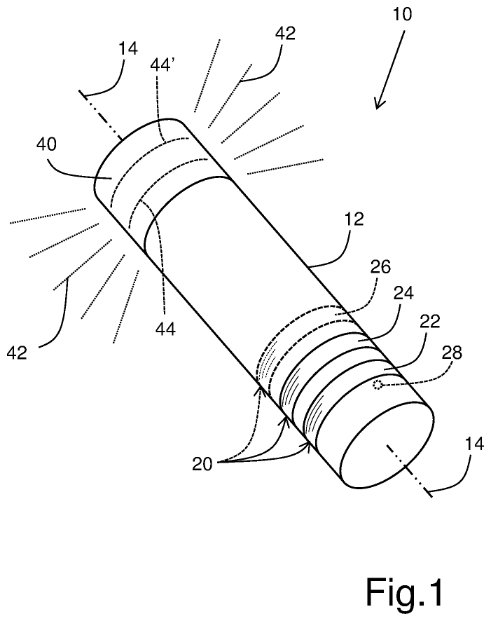

In FIG. 1, there is shown a schematic representation of an embodiment 10 of a sleep-inducing electronic device in accordance with the present invention. The device 10 is for being in skin contact with a user 100 and typically includes an input unit 20, a processor unit 30 and an output unit 40. The input unit 20 includes at least first 22 and second 24 sensors detecting physiological signals of the user 100 via the skin contact. In the present embodiment 10, the user 100 typically holds one longitudinal end of a preferably cylindrical housing 12 having the sensors 22, 24 located thereat, such that the devices 10 typically contacts the palm of the hand 102 of the user 100. The other longitudinal end of the housing 12 typically include the output unit 40 emitting a visual light stimulus signal.

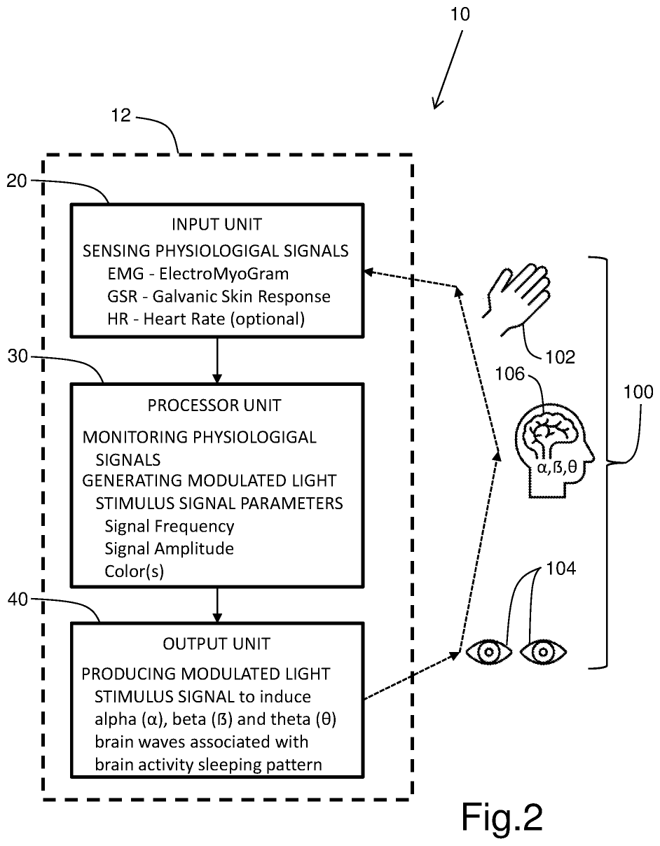

The processor unit 30 (not illustrated in FIG. 1 as it is preferably located inside the housing 12, but shown in FIG. 2) operatively connects to the first 22 and second 24 sensors to receive the detected physiological signals therefrom. The processor unit 30 monitors at least one physiological parameter, and preferably two physiological parameters from a parameter group consisting of an electromyogram (EMG) signal and a galvanic skin response (GSR) signal, and converts the at least one physiological parameter into light stimulus signal parameters. The output unit 40 operatively connects to the processor unit 30 to receive the generated light stimulus signal parameters therefrom and produces a corresponding light stimulus signal 42 for the eyes 104 of the user 100.

Typically, the parameter group further optionally includes a heart rate (HR) signal which could be sensed either via the first 22 and/or second 24 sensor, and/or via a third sensor 26 (indicated in stippled lines in FIG. 1) also operatively connected to the processor unit 30. The heart rate (HR), optionally considered in addition to the above at least one physiological parameter (EMG and/or GSR), could also be sensed by an infrared (IR) or other opto-electronic sensor 28 that could be located at any desired location on/inside the housing 12, but preferably around sensors 22, 24, 26 in proximity with the user's hand 102 holding the housing 12. The third sensor 26 could also be used as a neutral ground electrode to reduce electrical (or electric signal) interferences. All sensors 22, 24 (and 26 whenever applicable) have an electrically conductive surface to electrically detect the physiological signals. Although the sensors are shown as being annular in shape, one of ordinary skills in the art would readily understand that any shape, location and/or orientation could be considered without departing from the scope of the present invention.

Conveniently, the light stimulus signal parameters include a frequency parameter, an amplitude parameter and a color parameter. Depending on the type of output unit 40, the color parameter essentially refers to the either electromagnetic frequency range(s) of the desired emitted color(s) (via light emitting diodes (LEDs) or the like) or the color(s) of a filter located above a “white color” source emitter.

Typically, the processor unit 30 timely modulates the light stimulus signal parameters for the light stimulus signal 42 to induce and improve the production of sleeping waves in the brain 106 of the user 100 usually associated with a brain sleeping pattern. Although not detailed herein, the correlation used in the processor unit 30 is essentially applicable to everyone. Obviously, a more accurate correlation could be obtained for a specific individual based on applicable monitoring tests made to the individual to determine his/her specific brain behavior to different visual stimuli. This specific correlation is preferably calibrated in factory. Also, the output unit 40 could produce a mono-color light stimulus signal 42, as illustrated in FIG. 1, or be divided into a plurality of sections (as illustrated by stippled line section separators 44) to provide a multi-color light stimulus signal 42 (combination of colors).

Preferably, the processor unit 30 timely modulates the light stimulus signal parameters over typically three successive stages of sleep of the user 100. Consequently, the light stimulus signal 42 induces production of modulated alpha (α), beta (ß) and theta (θ) waves activity in the user's brain electroencephalogram (EEG) that will in turn be detectable by the controller unit 30 in the at least one physiological parameter monitored via the sensors 22, 24, 26.

Typically, the processor unit 30 also balances the detected physiological signals (EMG and/or GSR, and/or HR) to reject artefact noise coming from at least one of external/environmental electromagnetic radiations (EMR) and body movements of the user 100.

Typically, the processor unit 30 controls operation of the device 10 upon detection of the user's physiological signals via at least one of the first 22 and second 24 sensors (and third sensor 26 whenever present).

Preferably, as illustrated in FIG. 1, all of the input unit 20, the processor unit 30, and the output unit 40 are within a same (or common) housing 12, that is preferably shaped to comfortably fit within the hand 102 of the user 100, waterproof and shock resistant.

Conveniently, the device 10 includes a long-lasting power source (not shown—well known in the art) located within the housing 12 and electrically connected to the controller unit 30 for operation thereof, and other units 20, 40 the device 10 close-by. Although not preferred (for multiple reasons, such as a cumbersome recharging wire or the like), one of ordinary skill in the art would readily understand that rechargeable or replaceable battery or the like could be considered without departing from the scope of the present invention.

In FIG. 2, there is illustrated a schematic organigram of the operation of the device 10 of the present invention effectively performing a real-time biofeedback monitoring of the physiological signals (EMG and/or GSR, and/or HR) in order to produce a modulated light stimulus signal 42 that will progressively induce modulated alpha (α), beta (ß) and theta (θ) waves activity in the user's brain electroencephalogram (EEG) associated with brain activity sleeping pattern. Electrophysiology (including physiological signals related to EMG and GSR, and also HR) is correlated to the alpha (α), beta (ß) and theta (θ) waves of the EEG of the brain.

Although not illustrated herein, one of ordinary skill in the art would readily understand that the sleep-inducing electronic device of the present invention could be easily modified without departing from the scope of the present invention, such that the housing 12 combining all three units 20, 30, 40 could be divided into two (2) or three (3) physically separated parts with both the input 20 and output 40 units being operatively connected with the processor unit 30. In such a case, for example, the input unit 20 could be in contact with the skin of any other region/peripheral of the user 100, as the palm of a foot, and the output unit 40 could be a display located away from the user 100 while displaying the light stimulus signal 42 visible to the user 100. The processor unit 30 could be physically located between both or close to either one of the input 20 and output 40 units. In such cases, the unit(s) physically separated from processor unit 30 could communicate with the latter using either wired or wireless communication path, as well known in the art.

Although the present invention has been described with a certain degree of particularity, it is to be understood that the disclosure has been made by way of example only and that the present invention is not limited to the features of the embodiments described and illustrated herein, but includes all variations and modifications within the scope of the invention as hereinabove described and hereinafter claimed.

1. A sleep-inducing electronic device for being in skin contact with a user, the device comprising:
an input unit including first and second sensors detecting physiological signals of the user;
a processor unit operatively connecting to the first and second sensors to receive the detected physiological signals therefrom, the processor unit monitoring at least one physiological parameter from a parameter group consisting of an electromyogram (EMG) signal and a galvanic skin response (GSR) signal, the processor unit converting the at least one physiological parameter into light stimulus signal parameters; and
an output unit operatively connecting to the processor unit to receive the light stimulus signal parameters therefrom and producing a corresponding light stimulus signal for the user.

2. The device of claim 1, wherein the parameter group further including a heart rate (HR) signal, and the at least one physiological parameter further includes the heart rate (HR) signal.

3. The device of claim 1, wherein the input unit further includes a third sensor operatively connecting to the processor unit and either detecting physiological signals of the user or being a neutral ground electrode.

4. The device of claim 1, wherein the light stimulus signal parameters include a frequency parameter, an amplitude parameter and a color parameter.

5. The device of claim 1, wherein the processor unit timely modulating the light stimulus signal parameters to improve sleeping brain waves of the user associated with a brain sleeping pattern.

6. The device of claim 5, wherein the processor unit timely modulates the light stimulus signal parameters over three successive stages of sleep of the user.

7. The device of claim 1, wherein the processor unit balancing detected physiological signals to reject artefact noise from at least one of external electromagnetic radiations (EMR) and body movements of the user.

8. The device of claim 1, wherein the processor unit controlling operation of the device upon detection of the user's physiological signals.

9. The device of claim 1, wherein the light stimulus signal induces production of alpha (α), beta (ß) and theta (θ) waves activity in the user's brain electroencephalogram (EEG) detectable by the controller unit in the at least one physiological parameter.

10. The device of claim 3, wherein the third sensor is a neutral ground electrode.

11. The device of claim 3, wherein the third sensor is an infrared sensor.

12. The device of claim 3, wherein the third sensor is an infrared opto-electronic sensor.

13. The device of claim 1, wherein the input unit, the processor unit, and the output unit are within a housing.

14. The device of claim 13, wherein the input unit, the device includes a power source located within the housing and electrically connected to the controller unit for operation of the device.

15. The device of claim 1, wherein the device includes a power source electrically connected to the controller unit for operation of the device.
